(19) United States
(12) Patent Application Publication
(10) Pub. No.: US 2022/0001059 A1
(43) Pub. Date: Jan. 6, 2022

(54) DECONTAMINATION OF RESPIRATORY EQUIPMENT

(71) Applicant: KONINKLIJKE PHILIPS N.V., EINDHOVEN (NL)

(72) Inventors: Laura Beringer, Monroeville, PA (US); Jason Bryant, Erie, PA (US); Matthew Heidecker, Erie, PA (US); James Horne, Carlsbad, CA (US); William Moret, Monroeville, PA (US); Matthew Perun, Carlsbad, CA (US); Matthew William Skinner, Monroeville, PA (US); Corey Linden, Erie, PA (US); Dustin Clark, Erie, PA (US)

(21) Appl. No.: 17/338,838

(22) Filed: Jun. 4, 2021

Related U.S. Application Data
(60) Provisional application No. 63/047,327, filed on Jul. 2, 2020.
(60) Provisional application No. 63/117,058, filed on Nov. 23, 2020.

(51) Int. Cl. A61L 2/20; A61L 2/26; A61L 2/24

(52) U.S. Cl. CPC A61L 2/208; A61L 2/26; A61L 2202/24; A61L 2202/122; A61L 2/24

(57) ABSTRACT

Described herein are methods, devices and systems for decontaminating respiratory medical devices and equipment. Decontamination includes removing humidity from an interior of a respiratory equipment decontamination chamber after an interior thereof is sealed to exclude ambient air. The interior of the decontamination chamber is conditioned by introducing vaporized hydrogen peroxide (VHP) thereto to achieve a target concentration, which is maintained at a predetermined concentration for a decontamination period. The interior of the decontamination chamber is then aerated. Other examples are disclosed and claimed.

### CROSS REFERENCE TO RELATED APPLICATIONS

This application claims priority to U.S. Provisional Patent Application Ser. Nos. 63/047,327 and 63/117,058, filed on Jul. 2, 2020 and Nov. 23, 2020, respectively, the contents of which are hereby incorporated by reference in their entireties.

### FIELD OF THE INVENTION

The subject matter described herein generally relates to decontamination of respiratory equipment and certain examples of systems and methods for decontaminating respiratory equipment such as ventilators are provided.

### BACKGROUND OF THE INVENTION

Healthcare-associated infections (HAIs), e.g., surgical site infections, central line-associated bloodstream infections, catheter-associated urinary-tract infections, and ventilator-associated pneumonia, present significant challenges for effective treatment and often result in prolonged patient hospitalization, increased risk of morbidity, and increased incidence of mortality. Despite advances in preventing HAIs, nosocomial infections continue to result in a significant loss of life and healthcare costs. Ventilator-associated pneumonia (VAP), a type of pneumonia which develops in endotracheal-intubated patients prescribed mechanical ventilation, is a specific class of HAI that is associated with increased morbidity, mortality, and treatment cost. Risk factors for contracting VAP include intubation, extended duration of ventilation, and bacterial colonization of ventilator circuits.

Respiratory therapy devices such as ventilators are intricate medical devices comprising complex internal gas pathway geometries constructed from diverse materials, including metals, elastomers, and thermoplastics. Owing to tortuous internal gas pathways which are often too narrow to effectively decontaminate with traditional liquid reagents, reusable respiratory therapy devices such as ventilators can become contaminated with pathogens following patient use.

To date, no FDA-approved methods are available for decontamination or disinfection of the intricate gas pathways of respiratory medical devices, including ventilators, continuous positive airway pressure (CPAP) devices, bi-level therapy devices (BiPAPs), patient interface masks, patient circuits, patient circuit accessories (e.g., tubing), or other respiratory equipment. Other low-temperature, gaseous methods commonly used for medical device decontamination—processes based on ozone (e.g., the Keredusy® process offered by Medizin & Service GmbH), ethylene oxide, chlorine dioxide, and formaldehyde—are carcinogenic and present toxicological risks to patients. Additionally, these techniques have been studied and documented to degrade and/or corrode the sensitive electronics, metals and non-metallic materials which comprise respiratory medical devices, presenting safety concerns for patients and challenges related to retaining device performance.

### SUMMARY OF THE INVENTION

Various embodiments provide methods, devices and systems for decontaminating respiratory equipment using vaporized hydrogen peroxide (VHP) without harming sensitive internal components of the respiratory equipment. Decontamination includes removing humidity from an interior of a respiratory equipment decontamination chamber after an interior thereof is sealed to exclude ambient air. The interior of the decontamination chamber is conditioned by introducing VHP thereto to achieve a target concentration, which is maintained at a predetermined concentration for a decontamination period. The interior of the decontamination chamber is then aerated.

In summary, one embodiment provides a respiratory equipment decontamination device, comprising: a respiratory equipment decontamination chamber including an interior that is reversibly sealed to exclude ambient air; an inlet that provides entry of vaporized hydrogen peroxide (VHP); a dehumidifier operatively coupled to the interior; and a controller programmed to: receive a selection indicating one or more types of respiratory equipment to be decontaminated; operate the dehumidifier to remove humidity from the interior of the respiratory equipment decontamination chamber after the interior is sealed; conditions the interior by introducing VHP thereto to achieve a target concentration; maintain, based on the selection, VHP at a predetermined concentration for a decontamination period by introducing VHP to the interior; and aerate the interior by removing VHP from the interior to achieve an aeration target concentration of VHP.

Another embodiment provides a method for respiratory equipment decontamination, comprising: receiving a selection indicating one or more types of respiratory equipment to be decontaminated; conditioning the respiratory equipment by introducing dehumidified vaporized hydrogen peroxide (VHP) thereto to achieve a target concentration; maintaining, based on the selection, the dehumidified VHP at a predetermined concentration for a decontamination period by introducing the dehumidified VHP to the a gas pathway of the respiratory equipment; and aerating the respiratory equipment by removing VHP from the gas pathway to achieve an aeration target concentration of VHP.

A further embodiment provides a respiratory equipment decontamination system, comprising: a conduit that provides entry of dehumidified vaporized hydrogen peroxide (VHP) to a gas pathway of the respiratory equipment; the conduit comprising one or more fittings coupled to the conduit and complimentary to a gas pathway component of the respiratory equipment; and a controller programmed to: condition the gas pathway by introducing dehumidified VHP thereto to achieve a target concentration; maintain dehumidified VHP at a predetermined concentration for a decontamination period by introducing dehumidified VHP to the interior; and aerate the gas pathway to achieve an aeration target concentration of dehumidified VHP.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE DRAWINGS

FIG. 1 illustrates a perspective view of an example respiratory equipment decontamination system in accordance with an embodiment.

FIG. 2 illustrates a side view of an example respiratory equipment decontamination system in accordance with an embodiment.

FIG. 3 illustrates an example method of respiratory equipment decontamination in accordance with an embodiment.

FIG. 4A and FIG. 4B illustrate examples of H₂O₂ and relative humidity, respectively, over time during an example decontamination cycle in accordance with an embodiment.

FIG. 5 illustrates an example control system in accordance with an embodiment.

### DETAILED DESCRIPTION OF EMBODIMENTS

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the claims, but is merely representative of those embodiments.

Reference throughout this specification to “embodiment(s)” (or the like) means that a particular feature, structure, or characteristic described in connection with the embodiment is included in at least one embodiment. Thus, appearances of the phrases “according to embodiments” or “an embodiment” (or the like) in various places throughout this specification are not necessarily all referring to the same embodiment.

As used herein, the singular form of “a”, “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware. The term “about” or “approximately” as used herein includes conventional rounding of the last significant digit.

Furthermore, the described features, structures, or characteristics may be combined in any suitable manner in one or more embodiments. In the following description, numerous specific details are provided to give a thorough understanding of example embodiments. One skilled in the relevant art will recognize, however, that aspects can be practiced without one or more of the specific details, or with other methods, components, materials, etc. In other instances, well-known structures, materials, or operations are not shown or described in detail to avoid obfuscation.

Vaporized hydrogen peroxide (VHP), H₂O₂, is known as a compatible methodology for efficiently and effectively decontaminating and/or sterilizing surfaces of a variety of medical devices and healthcare facilities. The VHP methodology has been shown to eradicate numerous microorganisms, including bacterial spores, mycobacteria, fungi, bacteria, and viruses. As VHP is capable of penetrating and effectively decontaminating and/or sterilizing the internal surfaces and lumens of medical devices such as polyurethane catheters, polyethylene tubing, and endoscopes, this decontamination technique is an attractive candidate for addressing contamination of sensitive electronic medical devices. In fact, the United States Environmental Protection Agency (EPA) investigated the use of VHP decontamination for reprocessing electronic equipment in military and commercial applications and concluded that this process presented a low risk of corrosion or destruction to sensitive electronic materials.

Certain types of ventilators are more susceptible to contamination based on their pneumatic design. Blower-based ventilators which draw in and deliver ambient air to patients can become contaminated within the internal gas pathway. Single-limb ventilators are blower-based devices which operate with bidirectional flow. Bidirectional flow can allow the patient's exhaled breath to contaminate the gas pathway. In comparison, the inspiratory gas pathway of dual-limb ventilators is less susceptible to contamination as a result of a unidirectional pneumatic design. However, during certain alarm states such as over pressure relief, there is possibility of device contamination from the patient breathing gas entering the inspiratory gas pathway. The current strategy for contamination mitigation of the internal gas pathways of ventilators and other respiratory therapy devices is the use of in-line bacterial and viral filters, however these are not always mandated in labelling and are subject to the patient, healthcare professional, or end-user following appropriate protocols.

The internal gas pathways of respiratory therapy devices have previously been difficult to decontaminate owing to challenges related to component geometry, as well as material compatibility. Material incompatibility and degradation can diminish device performance and create biological risks to patients through the generation of harmful volatile organic compounds (VOCs). As limited techniques exist for decontaminating the internal gas pathway components of respiratory therapy devices, devices such as ventilators which have been used to treat patients with highly communicable disease (e.g., tuberculosis, severe acute respiratory syndrome, etc.) must currently be taken out of service to avoid the risk of further transmission.

The coronavirus disease 2019 (COVID-19) pandemic, caused by the spread of severe acute respiratory syndrome coronavirus 2 (SARS-CoV-2), is rapidly evolving. Globally, the COVID-2019 pandemic has resulted in significant loss of life and overwhelmed healthcare facilities. The SARS-CoV-2 virus is highly infectious, and there is currently a shortage of ventilator and other critical respiratory devices needed for treating incoming patients. This shortage has placed significant stress on healthcare systems, and strategies for addressing this scarcity are required. One potential strategy to alleviate the potential transmission of COVID-19 among patients in an already over-taxed health care system and discourage the potential for additional HAIs is to disinfect the gas pathway of ventilators and other respiratory equipment in between patients.

Owing to excellent material compatibility, low toxicity, and facile processing of vaporized hydrogen peroxide (VHP), a VHP-based process represents an appropriate methodology for decontaminating the internal gas pathways of respiratory medical devices such as ventilators, continuous positive airway pressure (CPAP) devices, bi-level therapy devices (BiPAPs), patient interface masks, patient circuits, patient circuit accessories (e.g., tubing), respirators, personal protective equipment (PPE), or other respiratory medical or therapy equipment (hereinafter collectively referred to as “respiratory equipment”). This decontamination process affords a solution for addressing risk factors associated with ventilator-associated pneumonia (VAP) transmission (i.e., suitable disinfection between patients with communicable diseases) and offers a solution for reducing healthcare-associated infection (HAI) rates. Furthermore, this decontamination technology provides a solution for addressing transmission of novel emerging pathogens and diseases (e.g., COVID-19), improving the clinical utility and efficacy of respiratory equipment.

Currently there are no marketed, FDA-approved methods for decontaminating, disinfecting, or sterilizing (collectively referred to as “decontaminating”) the gas pathways of ventilators. Described herein are systems and methods for placing respiratory equipment such as a ventilator into a decontamination chamber and using VHP to disinfect the entirety of the respiratory equipment, including the internal gas pathway(s), its exterior, as well as internal compartments. The biocompatibility and toxicological safety of respiratory equipment decontaminated using the VHP processes described herein were evaluated using ISO 18562-2:2017 and 18562-3:2017 methodologies to determine and risk assess all potentially generated volatile organic compounds (VOCs) to confirm patient safety.

The description now turns to the figures. The illustrated example embodiments will be best understood by reference to the figures. The following description is intended only by way of example and simply illustrates certain selected example embodiments representative of the invention, as claimed.

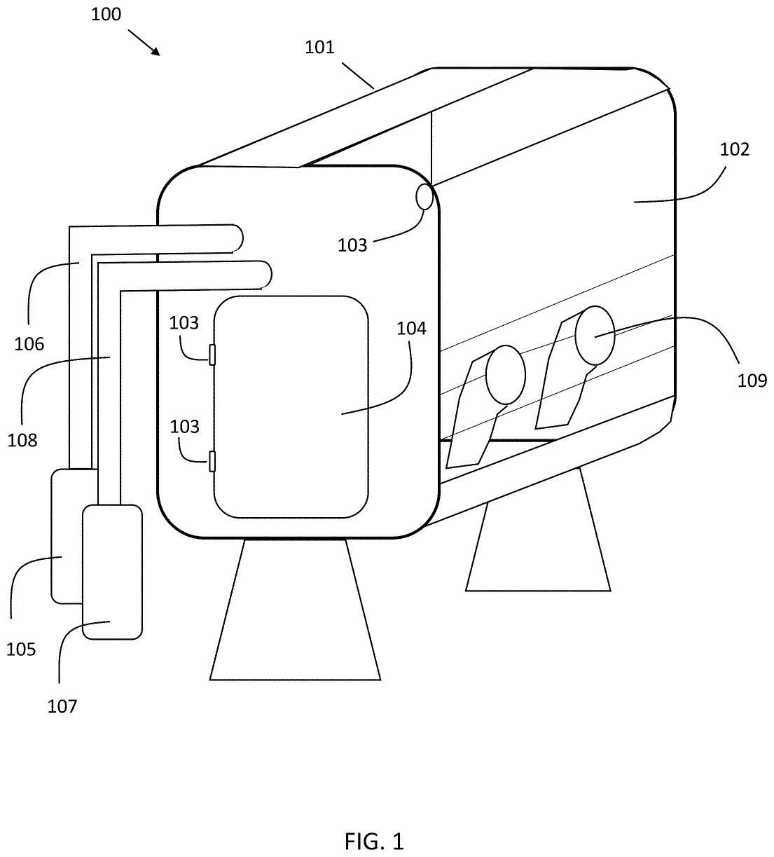

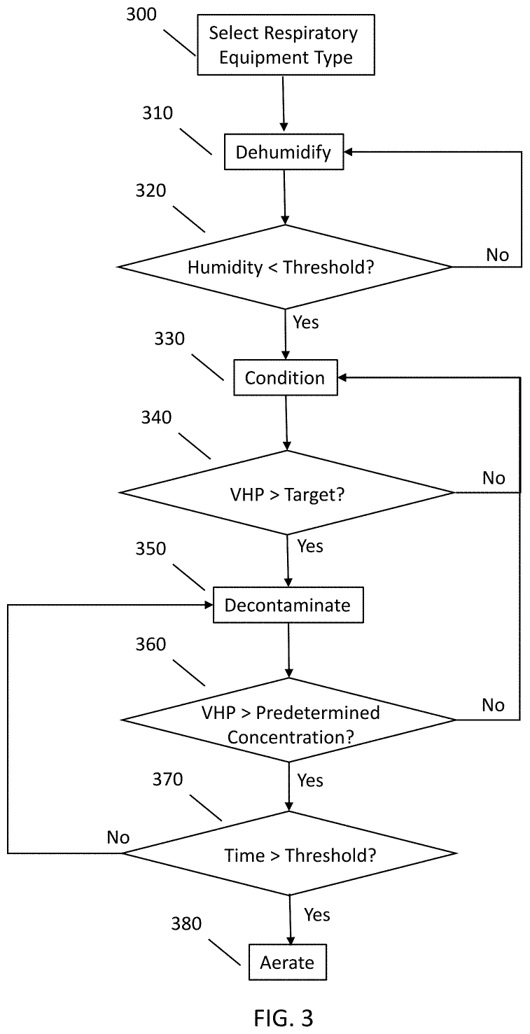

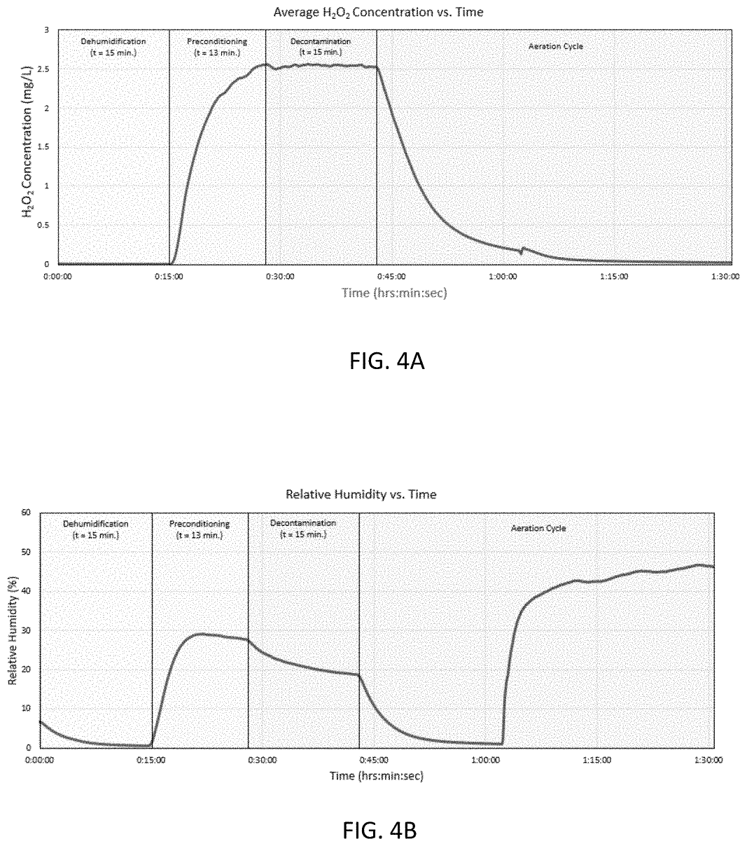

Referring to FIG. 1, an embodiment provides a system 100 for decontaminating respiratory equipment such as ventilators. In an embodiment there are four main stages involved in the VHP decontamination of respiratory equipment that is placed inside of an enclosed decontamination chamber: (1) dehumidification; (2) conditioning or preconditioning; (3) decontamination; and (4) aeration, as further described in connection with FIG. 3 as well as FIG. 4A and FIG. 4B.

As illustrated in FIG. 1, an embodiment provides a decontamination chamber 101 into which a ventilator or other respiratory equipment or articles that are to be decontaminated are placed. The interior of the decontamination chamber may be varied to suit a particular context. In an example embodiment designed to decontaminate respiratory equipment such as a patient ventilator, e.g., blower-based ventilators such as the PHILIPS Respironics Inc. EV300 and V60 ventilators, the interior of the decontamination chamber 101 may be about 50 ft³.

One or more ventilators and/or other respiratory equipment may be placed into the interior of the decontamination chamber 101 via an opening 104, which may be attached to a lateral side of the decontamination chamber 101 via hinges 103 or other suitable mechanism. Additionally or alternatively, another opening may be provided, such as by attaching a front side or panel 102 to the decontamination chamber 101 via a hinge 103 or like mechanism. In an example embodiment, various panels or sides, e.g., 102, may be made of transparent material such as plastic or glass to afford a view of the interior of the decontamination chamber 101. Further, the decontamination chamber 101 may take the form of a glovebox, as indicate din FIG. 1, where one or more gloves 109 are provided to permit manual manipulation of a ventilator or other item positioned in the interior of the decontamination chamber 101. For example, a user may use gloves 109 to turn a ventilator on or off when positioned inside the decontamination chamber 101 to allow circulation of VHP rich air through the ventilator's interior.

In an embodiment, while the decontamination chamber 101 may be operated at ambient temperature and pressure, it is sealed to exclude the external environmental air from the interior space of the decontamination chamber 101. This permits the dehumidification, conditioning and maintenance of sufficient VHP concentration within the interior.

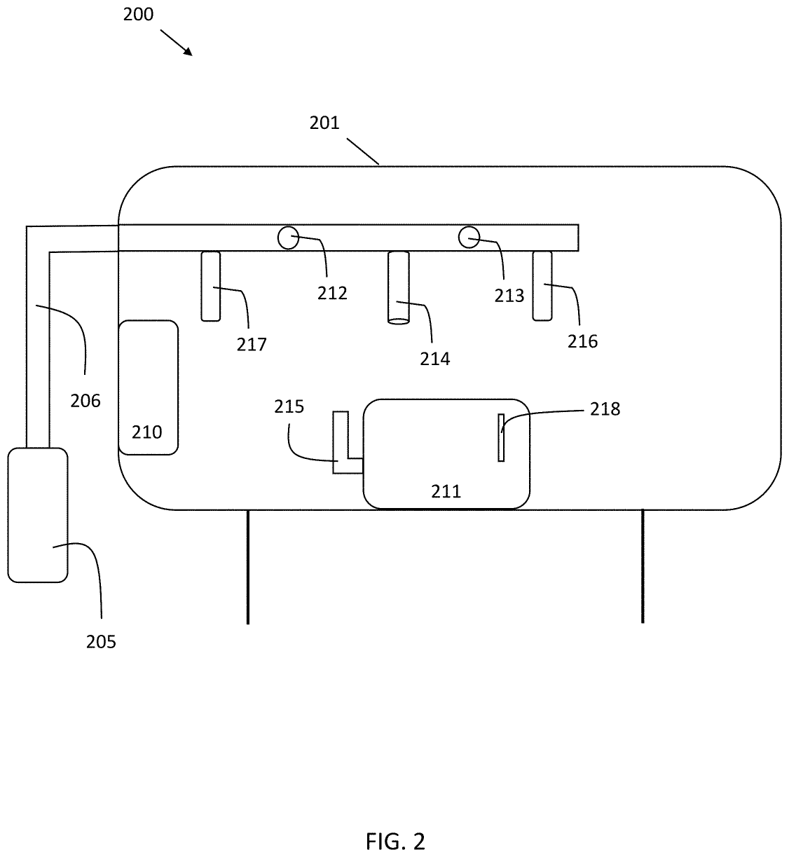

In a dehumidification phase, by way of example, the humidity of the decontamination chamber 101 is reduced through a dehumidification process of approximately 10 to 20 minutes. Depending on the volume of the interior of the decontamination chamber 101 this time may be as short as 1 minute (for very small volumes) or as long as 24 hours (for a large room). As shown in the example illustration of FIG. 2, active sensors 216, 217 are maintained within the decontamination chamber 201 to obtain data related to the appropriate concentration of relative humidity (RH). In one example embodiment, sensors 216, 217 may be VAISALA HPP272 PEROXCAP Humidity, Temperature and Vaporized H₂O₂ meters. In an example embodiment, three or more sensors such as sensors 216, 217 may be placed in the interior of the decontamination chamber. The sensor data may be provided to an integrated or remotely located controller (as further described herein) and used to adjust the dehumidification or other processes. Ranges of acceptable RH for decontamination of sensitive electronics equipment, such as that included in a ventilator, could include values of 5-40%. These values may be dependent upon volume of the decontamination chamber 101 and an optimal range may be established dependent on the number or type of respiratory equipment units being decontaminated or the nature of the other item(s) placed into the decontamination chamber 101 to be decontaminated.

As shown in the example of FIG. 2, a dehumidifier 210 may be located within the interior of the decontamination chamber 201. The dehumidifier 210 may be controlled via feedback from sensors 216, 217, via feedback from integrated sensors within the dehumidifier 210 itself, or a suitable combination of the foregoing. In one example, the dehumidifier 210 may be incorporated into another unit or component, e.g., a dehumidifier 210 may be provided as part of the VHP source 205.

Referring back to the example illustration provided in FIG. 1, in a conditioning phase, VHP is introduced to the decontamination chamber 101 from a source of VHP 105, e.g., via a conduit or tubing 106 that leads into the interior of the decontamination chamber. In an embodiment, the source of VHP may include a blower assembly that transmits VHP via conduit 106 into the interior of the decontamination chamber 101. One example of a suitable source of VHP 105 is the STEMS VHP 1000ED vaporized hydrogen peroxide generator. An embodiment may use compressed gasses (e.g., air and oxygen) as the source instead of using a blower-based ambient air source, or these techniques may be combined.

Referring again to FIG. 2, the conduit or tubing 206 may extend into the interior of the decontamination chamber 201 and provide a structure or attachment point for other equipment, such as sensors 216, 217. By way of example, in an embodiment that includes complimentary fitting(s) 214 or attachment(s) for particular equipment, e.g., a fitting 214 may be complimentary to and configured to releasably attach or couple to a gas pathway component 215 of a ventilator 211, such may be provided via conduit or tubing 206, e.g., via flexible tubing or like attachment or coupling, to facilitate direct introduction of VHP into a particular machine or component thereof. In an example, a fitting 214 may include a keyed interface that compliments the interface of a gas pathway component 215 of a ventilator 211. In an embodiment, a fitting 214 that interfaces with a gas pathway component 215 may remove the need to include a decontamination chamber 201 such that dehumidified VHP is introduced into the gas pathway in a controlled, closed-loop manner and does not require the respiratory equipment 211 to be housed or sealed with a decontamination chamber 201. In other words, as with other embodiments where certain components are omitted, added or rearranged, an embodiment may be adapted to omit the decontamination chamber 201 from the system in favor of a direct connection of conduit 206 to a gas pathway component 215, e.g., via a fitting of connection, such as fitting 214.

It is noted that even in an embodiment that does not include a fitting 214, a ventilator 211 may have VHP introduced into its interior pathways and throughout via suitable concentration of VHP within the interior of the decontamination chamber 101 and operating the ventilator 211 while in the VHP rich interior of the decontamination chamber 101. In this regard, the conduit or tubing 206 may provide inlets or openings 212, 213 for the general introduction of VHP into the interior of the decontamination chamber 201, which may contain one or more ventilators 211 or other respiratory equipment such as personal protective equipment (PPE). While a separate aeration unit 107 and associated conduit or tubing 108, and separate dehumidifier 210, are illustrated in FIG. 1 and FIG. 2, it is noted that these components and/or their functionality may be combined with VHP source 105 and conduit or tubing 106, e.g., dehumidification and/or VHP removal may take place via inlets or openings 212, 213, in which case these may act as exhausts or outlets.

An embodiment may operate to condition the interior of the decontamination chamber 101 to a target concentration of VHP. The target VHP concentration may be equal to the VHP concentration maintained throughout the decontamination cycle, above this concentration, or below this concentration. In one example, the target VHP concentration is about 2.3 mg/liter. The injection or introduction rate of VHP is controlled to reach a desired target VHP concentration, which may be input by a control interface or selected based on a predetermined program designed for given types of item(s) or object(s) to be decontaminated, such as a ventilator, PPE, etc. By way of specific example, an operator may input a numeric target VHP concentration or an operator may input a selection of respiratory equipment or object type, which is matched to a predetermined decontamination routine that includes an appropriate target VHP concentration.

The target concentration of VHP is to be achieved throughout the decontamination space, as confirmed for example with one or more indicators, e.g., via data feedback of one or more sensors such as sensors 216, 217 located within the interior of the decontamination chamber 201. The concentration desired may be dependent upon the size of the chamber's interior and can be scaled down to approximately 1.0 mg/liter or less for larger chambers or scaled up to approximately 3.5 mg/liter for smaller chambers.

In one example, the conditioning phase for respirator decontamination lasts one (1) to sixty (60) minutes, depending on the volume within the decontamination space. In the specific examples of FIG. 4A-B, the dehumidification, conditioning/preconditioning, and decontamination phases each last about 15 minutes. As with other parameters of a decontamination process, the timing of the conditioning phase, as with other phases, may be modified for different chambers, different respiratory equipment, different reduction targets, or combinations thereof. In some cases, the timing of the conditioning phase or other phases may be dynamically modified, e.g., based on user input or automatically, such as based on sensor feedback data.

After a target conditioning concentration and RH are achieved within the decontamination chamber 101, a decontamination phase may be provided. During the decontamination phase, the VHP concentration throughout the interior of the decontamination chamber 101 is maintained at a predetermined concentration or within a predetermined concentration range. In one example, the predetermined concentration maintained in the decontamination phase is about 2.3 mg/liter. In an embodiment, the VHP concentration may be as low as 0.6 mg/L and maintained in excess of 10 minutes, or as high as 3.4 mg/L and maintained less than 1 minute. In an embodiment, a predetermined VHP concentration or concentration range is maintained for a predetermined duration. In an embodiment, one or more of the VHP concentration (or concentration range) and the duration may be modified dynamically. For example, a lower VHP concentration may extend the duration of the decontamination phase. Similarly, sensors such as sensors 216, 217 may adjust the VPH input to modify the VHP concentration, modify the duration of the decontamination phase, or a combination of the foregoing, based on data obtained by the sensors. Other data available to the system may also be used for such modifications, such as user supplied selections of equipment types, fittings 215 identified (automatically or via user input) as attached to ventilator(s) 211 or a component thereof 215, operating status of a ventilator 211, etc.

In an embodiment, a VHP decontamination phase is sufficient to achieve a 6-log reduction of a standard contaminate such as biological indicator (BI) organism like Geobacillus stearothermophilus. Decontamination within the range of VHP 0.6 mg/L and 3.4 mg/L concentration has been confirmed to deliver a 6-log reduction, which is typically referenced as an appropriate level of disinfection for reusable health care devices per Association for the Advancement of Medical Instrumentation (AAMI) and Centers for Disease Control and Prevention (CDC) guidance. The decontamination time or duration may be as short as reasonable to deliver a 6-log reduction. An example duration is about 30 minutes or less. The examples of FIG. 4A-B illustrate a decontamination phase lasting about 15 minutes. Optionally, embodiments may use a duration from about 1 to about 600 minutes, depending on the number of respiratory devices, their type, the nature of the decontamination chamber 101 (e.g., fittings 215 available and in use), the volume of the chamber being utilized, the concentration of VHP, or combinations thereof.

Following a decontamination phase, an embodiment may provide an aeration phase in which VHP is removed from the interior of the decontamination chamber 101. In an example aeration process, VHP is actively converted on removal of the VHP from the decontamination chamber 101. By way of example, an aeration unit 107 may be coupled to the decontamination chamber 101 via a conduit or tubing 108 and extract VHP from the interior of the decontamination chamber 101, e.g., via use of a fan. Active conversion via a catalytic converter within aeration unit 107 may be provided, e.g., via conversion of VHP to CO₂ and/or water. Aeration unit 107 may exchange the VHP with ambient air. The rate of aeration and the duration of the aeration phase may be dependent on the size and number of respiratory equipment devices contained within the decontamination chamber 101. In one example, the aeration phase may take approximately 30-60 minutes. In the examples of FIG. 4A-B, the aeration phase is about 45 minutes. The aeration phase may be actively monitored similar to the other phases, e.g., the aeration phase may be completed once internal sensors such as sensors 216, 217 read 1.0 ppm or less of VHP (the OSHA permissible exposure limit of residual VHP).

Referring now to FIG. 3, examples of operations of the foregoing systems and devices will now be provided. FIG. 4A and FIG. 4B illustrate examples of VHP concentration and relative humidity, respectively, during an example decontamination cycle for ventilators Trilogy EVO and V60. Further, the example VHP concentration (H₂O₂ mg/L) and timing (hrs:min:sec) illustrated in FIG. 4A, and relative humidity (%) and timing (hrs:min:sec) illustrated in FIG. 4B, are an example of appropriate decontamination of ventilators within a decontamination chamber 101 as described herein to achieve a 6-log reduction in a BI organism Geobacillus stearothermophilus. The examples of FIG. 4A and FIG. 4B may be referred to in connection with FIG. 3 to illustrate example VHP concentrations and relative humidity values as well as example cycle timing for decontaminating respiratory equipment in the interior of a decontamination chamber 101. In FIG. 4A-B, a dehumidification phase is 15 minutes, a preconditioning phase is 13 minutes, and a decontamination phase is 15 minutes, with the aeration phase lasting 47 minutes.

In an embodiment, by way of example, a ventilator is received and placed into a decontamination chamber 101. The decontamination chamber 101 may be a discrete six-sided box of varying volume, a bio-decontamination chamber of varying internal volume, a room within a biosafety laboratory, or other construct intended to safely contain VHP for decontaminating a respiratory equipment device.

The interior of the decontamination chamber 101 is sealed to prevent VHP from escaping into the ambient atmosphere and to allow control of the VHP concentration within the decontamination chamber 101 to achieve the target concentration as well as for easy maintenance thereof for the duration of the decontamination and aeration phases. An operator may provide a selection or other indication as to the type of respiratory equipment type to be contaminated at 300.

In an example decontamination process, a cycle begins with dehumidifying the relative humidity to a target value or range in a dehumidifying step 310. An embodiment operates a dehumidifier as indicated at 310 to achieve a relative humidity of between 0% and 20%. Other embodiments may use different relative humidity, e.g., from 20% to 60%, depending on the nature of the items to be decontaminated. As with other processes described herein, the humidity level or target amount may be manually input or indicated by an operator, e.g., as a percentage or may be selected automatically in response to item identification (e.g., PPE, ventilator type or model, etc.).

As will be appreciated by those having ordinary skill in the art, the dehumidifying, conditioning, decontaminating, and aerating parameters are dependent upon the operating conditions, e.g., the internal volume of the decontamination chamber 101, items to be decontaminated, etc., and are only required to deliver a predetermined reduction, e.g., 6-log reduction of a model BI organism. An embodiment may be configured to operate the dehumidifying, conditioning, decontaminating, and aerating phases to be the shortest duration cycle(s) that are reasonable to deliver consistent reduction of the desired magnitude, e.g., a 6-log reduction.

Adjustment of the overall process or any particular phase may be based on the chamber size, environmental conditions, and load (e.g., number of contaminated devices or articles being decontaminated, the nature thereof, etc.). For example, the following parameters yielded a 6-log reduction of a BI organism without altering material chemistry or VOC profile of ventilators that would pose a toxicological risk to the patient: 48 ft³ chamber; Humidity <30%; up to 15 ventilators; 2.3 mg/Liter VHP; with running times of 15 minute dehumidification, 12 minute conditioning, 75 minute decontamination, and up to 90 minutes aeration.

Responsive to a determination at 320 that the relative humidity is below a threshold amount, e.g., below 30%, an embodiment transitions to a conditioning phase at 330. Otherwise, the dehumidification may continue as indicated at 310. The conditioning at 330 includes introducing VPH into the interior of the decontamination chamber 101. The sensed concentration of VHP is compared to a target concentration, as indicated at 340. If the VHP concentration has achieved the target concentration, e.g., 2.3 mg/Liter, as determined at 340, the process may transition to a decontamination phase, as indicated at 350. Otherwise, further VHP is introduced into the interior of the decontamination chamber 101.

The decontamination phase 350 continues for a specified time, e.g., to achieve a predetermined reduction, such as a 6-log reduction of a BI organism. For example, the decontamination phase may include programmatically comparing (e.g., at predetermined or dynamically determined intervals) the concentration of VHP to a predetermined concentration or concentration range, as indicated at 360. If the concentration of VHP is below the predetermined concentration, e.g., lower than the predetermined concentration but within acceptable range, more VHP may be introduced. If the VHP concentration within the interior of the decontamination chamber 101 is at or above the predetermined concentration, as determined at 360, and the decontamination phase timer has not expired, as determined at 370, the decontamination phase continues, as indicated in the example of FIG. 3. In contrast, if the concentration of VHP has been maintained at or above the predetermined concentration, as determined at 360, and the decontamination phase timer has expired, as determined at 370, the process transitions to the aeration phase at 380 during which VHP is removed and/or neutralized to return the interior of the decontamination chamber 101 to approximately the ambient environment.

The VHP decontamination of ventilators and other respiratory equipment may also include a series of quality control checks to ensure that decontamination is occurring and a real-time VHP concentration is measured. By way of example, sensors such as sensors 216, 217 may report in real-time the VHP concentration in the interior of the decontamination chamber 201. An alarm, indication or alert may be provided if the VHP concentration is within a predetermined range, outside of a predetermined range, or a suitable combination of indicators, alerts, etc. may be provided. By way of example, a display panel or other output device associated with the system 100 or a sub-component, e.g., sensors 216, 217, may provide a visible or audible indication or alert. In addition to or as an alternative to sensors such as active sensors 216, 217, other indicators may be utilized. In one example, a passive indicator 218 such as chemical indicator tape for confirming VHP permeation or one or more biological indicators (BI) to confirm reduction may be placed in strategic positions either within the decontamination chamber 101 or in/on the device to be decontaminated, e.g., ventilator 211, to ensure VHP penetration and confirm at least a 6-log reduction of a model BI organism.

As described herein, an embodiment may utilize fittings 214 or custom conduits to integrate into the gas pathway interface 215 of a medical device such as a ventilator 211 to deliver the VHP in a closed loop through the gas pathway. This option may be used alone to offer a smaller package that isolates decontaminating VHP to just the gas pathway and does not expose the other electrical and mechanical components to the VHP, and in that respect functions similarly to existing processes based on ozone. Additionally or alternatively, such an arrangement may be used in combination with the decontamination chamber 101, e.g., which additionally provides general decontaminating VHP to the interior of the decontamination chamber 101, e.g., via inlets 212, 213. In an embodiment, a compressed source of VHP may be utilized and in order to expose the compressed gas pathways to the VHP, a high pressure canister may be used to inject the VHP into the compressed gas pathways.

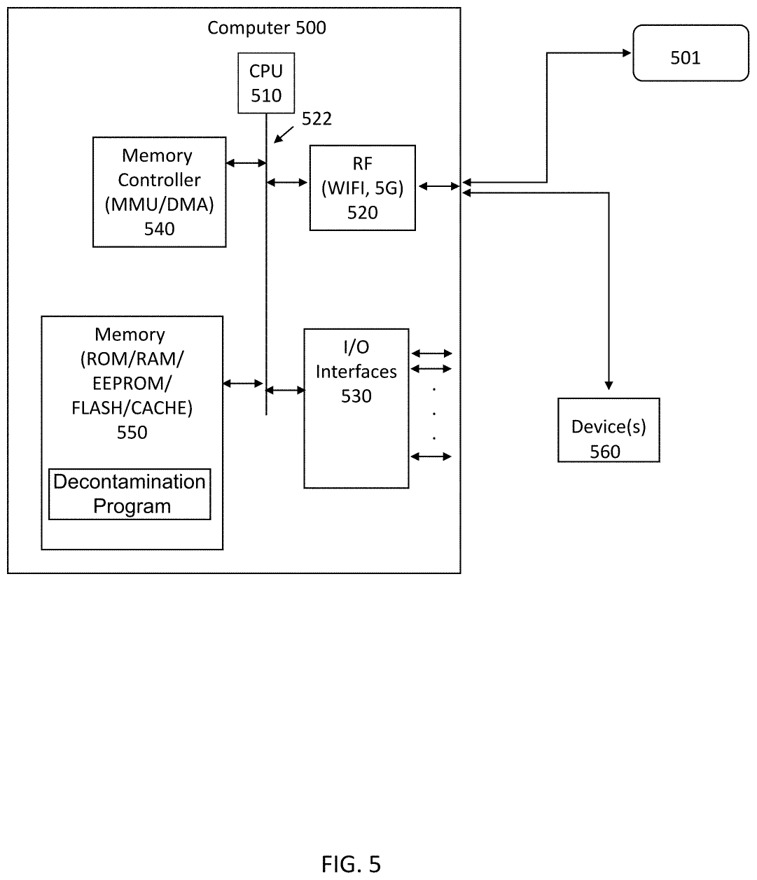

Referring to FIG. 5, it will be readily understood that certain embodiments can be implemented using any of a wide variety of devices or combinations of devices. In FIG. 5 an example system on chip (SoC) included in a computer 500 is illustrated, which may be used in implementing one or more embodiments, e.g., as a controller. The SoC or similar circuitry outlined in FIG. 5 may be implemented in a variety of devices, for example similar circuitry may be included in a VHP source device 105, 205 or another device or platform. In addition, circuitry other than a SoC, an example of which is provided in FIG. 5, may be utilized in one or more embodiments. The SoC of FIG. 5 includes functional blocks, as illustrated, integrated onto a single semiconductor chip to meet specific application requirements.

The central processing unit (CPU) 510, which may include one or more graphics processing units (GPUs) and/or micro-processing units (MPUs), includes an arithmetic logic unit (ALU) that performs arithmetic and logic operations, instruction decoder that decodes instructions and provides information to a timing and control unit, as well as registers for temporary data storage. The CPU 510 may comprise a single integrated circuit comprising several units, the design and arrangement of which vary according to the architecture chosen.

Computer 500 also includes a memory controller 540, e.g., comprising a direct memory access (DMA) controller to transfer data between memory 550 and hardware peripherals. Memory controller 540 includes a memory management unit (MMU) that functions to handle cache control, memory protection, and virtual memory. Computer 500 may include controllers for communication using various communication protocols (e.g., I²C, USB, etc.).

Memory 550 may include a variety of memory types, volatile and nonvolatile, e.g., read only memory (ROM), random access memory (RAM), electrically erasable programmable read only memory (EEPROM), Flash memory, and cache memory. Memory 550 may include embedded programs and downloaded software, e.g., control software such as a decontamination program for operating VHP source device 105, 205, aeration unit 107, ventilator 211, etc. By way of example, and not limitation, memory 550 may also include an operating system, application programs, other program modules, and program data, which may be downloaded, updated, or modified via remote devices such as device(s) 560.

A system bus 522 permits communication between various components of the computer 500. I/O interfaces 530 and radio frequency (RF) devices 520, e.g., WIFI and telecommunication radios, may be included to permit computer 500 to send and receive data to and from remote devices using wired or wireless mechanisms. The computer 500 may operate in a networked or distributed environment using logical connections to one or more other remote computers or databases. The logical connections may include a network, such local area network (LAN) or a wide area network (WAN), but may also include other networks/buses. For example, computer 500 may communicate data with and between a VHP source device 105, 205 and other devices, e.g., a decontamination chamber 501 (or components thereof such as sensors 216, 217), remote device(s) 560 that persistently store logs or reports of decontamination cycles or provide programs or updates to computer 500, etc.

The computer 500 may therefore execute program instructions configured to store and analyze sensor data reporting on conditions within a decontamination chamber 501, and perform other functionality of the embodiments, as described herein. A user can interface with (for example, enter commands and information) the computer 500 through input devices, which may be connected to I/O interfaces 530. A display or other type of device may be connected to the computer 500 via an interface selected from I/O interfaces 530.

It should be noted that the various functions described herein may be implemented using instructions stored on a memory, e.g., memory 550, that are transmitted to and executed by a processor, e.g., CPU 510. Computer 500 includes one or more storage devices that persistently store programs and other data. A storage device, as used herein, is a non-transitory computer readable storage medium. Some examples of a non-transitory storage device or computer readable storage medium include, but are not limited to, storage integral to computer 500, such as memory 550, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions (computer code) may be provided to a processor of a device to produce a special purpose machine, e.g., a controller, such that the instructions, which execute via a processor of the device implement the functions/acts specified.

It is worth noting that while specific elements are used in the figures, and a particular ordering of elements has been illustrated, these are non-limiting examples. In certain contexts, two or more elements may be combined, an element may be split into two or more elements, or certain elements may be re-ordered, re-organized, or omitted, as appropriate, as the explicit illustrated examples are used only for descriptive purposes and are not to be construed as limiting.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A respiratory equipment decontamination device, comprising:
a respiratory equipment decontamination chamber including an interior that is reversibly sealed to exclude ambient air;
an inlet that provides entry of vaporized hydrogen peroxide (VHP);
a dehumidifier operatively coupled to the interior; and
a controller programmed to:
receive a selection indicating one or more types of respiratory equipment to be decontaminated;
operate the dehumidifier to remove humidity from the interior of the respiratory equipment decontamination chamber after the interior is sealed;
condition the interior by introducing VHP thereto to achieve a target concentration;
maintain, based on the selection, VHP at a predetermined concentration for a decontamination period by introducing VHP to the interior; and
aerate the interior by removing VHP from the interior to achieve an aeration target concentration of VHP.

2. The device of claim 1, wherein the interior of the respiratory equipment decontamination chamber is about 48 ft³.

3. The device of claim 1, wherein the target concentration and the predetermined concentration are equal.

4. The device of claim 1, wherein the predetermined concentration is about 0.6 mg/L to about 3.4 mg/L.

5. The device of claim 1, wherein the predetermined concentration is about 2.3 mg/L.

6. The device of claim 1, wherein the decontamination period is about 1 minute to about 600 minutes.

7. The device of claim 1, wherein the controller is programmed to operate the dehumidifier to achieve a humidity for the interior of about 5% to about 40% relative humidity.

8. The device of claim 1, wherein the selection indicates one or more ventilators.

9. The device of claim 1, wherein the aeration target concentration is about 1.0 ppm.

10. The device of claim 1, comprising one or more indicators;
the one or more indicators providing an indication of a concentration of VHP within the interior;
the controller being programmed to maintain the VHP based on the indication.

11. A method for respiratory equipment decontamination, comprising:
receiving a selection indicating one or more types of respiratory equipment to be decontaminated;
conditioning the respiratory equipment by introducing dehumidified vaporized hydrogen peroxide (VHP) thereto to achieve a target concentration;
maintaining, based on the selection, the dehumidified VHP at a predetermined concentration for a decontamination period by introducing the dehumidified VHP to a gas pathway of the respiratory equipment; and
aerating the respiratory equipment by removing the dehumidified VHP from the gas pathway to achieve an aeration target concentration of VHP.

12. The method of claim 11, wherein the predetermined concentration is about 2.3 mg/L.

13. The method of claim 11, wherein the selection indicates one or more ventilators.

14. The method of claim 11, comprising receiving data from one or more indicators;
the data providing an indication of a concentration of dehumidified VHP within the gas pathway;
wherein the maintaining is based on the data received from the one or more indicators.

15. A respiratory equipment decontamination system, comprising:
a condui that provides entry of dehumidified vaporized hydrogen peroxide (VHP) to a gas pathway of the respiratory equipment;
the conduit comprising one or more fittings coupled to the conduit and complimentary to a gas pathway component of the respiratory equipment; and
a controller programmed to:
condition the gas pathway by introducing dehumidified VHP thereto to achieve a target concentration;
maintain dehumidified VHP at a predetermined concentration for a decontamination period by introducing dehumidified VHP to the gas pathway; and
aerate the gas pathway by removing dehumidified VHP from the gas pathway to achieve an aeration target concentration of dehumidified VHP.
